(19) United States
(12) Patent Application Publication
(10) Pub. No.: US 2009/0182314 A1
(43) Pub. Date: Jul. 16, 2009

(54) WORKING TOOL FOR LASER-FACILITATED REMOVAL OF TISSUE FROM A BODY CAVITY AND METHODS THEREOF

(76) Inventors: Eliahu Eliachar, Haifa (IL); Dan Sade Hochstadter, Kibbutz Bet Alfa (IL); Yosef Gross, Moshav Mazor (IL); Ofer Yossepowitch, Petach Tikva (IL); Nir Lilach, Kfar Yehoshua (IL); Ram Grossfeld, Haifa (IL); Arnon Moshayof, Tel Aviv (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/120,303

(22) Filed: May 14, 2008

Related U.S. Application Data
(60) Provisional application No. 61/021,037, filed on Jan. 15, 2008.
(60) Provisional application No. 61/048,240, filed on Apr. 28, 2008.

(51) Int. Cl. A61B 18/20

(52) U.S. Cl. 606/15

(57) ABSTRACT

Apparatus is provided, including an intraluminal tube having a proximal end and a distal end, the tube being shaped to provide a longitudinal lumen. A treatment element, having a proximal end and a distal end, is disposed within the lumen of the tube. A handle assembly is coupled to the proximal end of the tube. A user offset controller is coupled to the handle assembly, and is configured to set an offset of the distal end of the treatment element with respect to the distal end of the tube. Other embodiments are also described.

### CROSS-REFERENCES TO RELATED APPLICATIONS

This application claims priority from U.S. Provisional Patent Application No. 61/021,037, filed on Jan. 15, 2008 and U.S. Provisional Patent Application No. 61/048,240, filed on Apr. 28, 2008, which are incorporated herein by reference.

### FIELD OF THE INVENTION

The present invention generally relates to a working tool having means to actuate a laser emitting effecter. More specifically, the invention pertains to laser-facilitated removal of tissue from a body cavity or lumen.

### BACKGROUND OF THE INVENTION

Over the last twenty years, endoscopic medical devices for removing tissue from internal body cavities have been developed in which the tissue removal is carried out by a laser beam. The laser beam is used to vaporize or ablate the tissue to be removed. The primary advantages of the use of a laser beam over analogous mechanical devices are that the cutting area and depth can be precisely controlled; that the laser itself simultaneously removes and coagulates the tissue of interest; U.S. Pat. Nos. 4,955,882; 5,061,266; 5,201,731; and 5,312,399 disclose a device in which resection and coagulation of tissue is accomplished by a beam of light emanating from a laser generator and directed to the tissue of interest by means of a fiber optic wave guide, while a mechanical cutting blade downsizes the pieces of tissue removed by the laser beam to allow removal by aspiration. U.S. Pat. Nos. 5,498,258; 5,334,183; and 6,152,919 disclose devices that combine laser and mechanical resection technologies: in the first a laser beam is used instead of an electrical current to heat a mechanical cutting blade, while the latter two comprise both a laser and a mechanical blade.

U.S. Pat. No. 7,300,447 to Eliachar et al., which is incorporated herein by reference, describes a working tool for a resectoscope, adapted for side-to-side resection of biological tissue using predetermined lateral movement. The resectoscope is adapted for either cold or hot resection and is either flexible or rigid. A suitable cutting member assembly and a method useful for lateral resection are also provided.

U.S. Pat. No. 4,955,882 to Hakky, which is incorporated herein by reference, describes a resectoscope for prostate surgery which includes a rotating cutting element mounted within an outer sheath adapted to be inserted into the urethra. The cutting element has helical threads along the length thereof and a cutting blade at its distal end. The outer sheath has a covered distal end portion which extends beyond and over the cutting blade and has an opening therethrough adjacent the cutting blade. Within the outer sheath is an inner sheath surrounding the cutting element except for the cutting blade. A fiber optic laser filament connected to a laser generator is positioned within the space between the inner and outer sheaths and extends along the length of the inner sheath to a position adjacent the cutting blade. The optic filament is surrounded by a third sheath and is adapted to be moved by the rotation of the cutting element so that the laser light beam from the optic filament advances through tissue to cut and coagulate the resected area before the cutting blade of the cutting element reaches the resected tissue. Irrigation fluid is provided to the area between the inner and outer sheaths and is withdrawn through the inner sheath. A telescope is also provided through the cutting element for viewing the area being resected. There is further provided use of ultrasound in conjunction with the laser resectoscope to plot the area of the prostate to be removed and with the assistance of a computer to control the operation of the laser to prevent cutting of tissue beyond the area of tissues to be removed.

U.S. Pat. No. 5,820,009 to Melling, et al., which is incorporated herein by reference, describes an articulated surgical instrument for use in laparoscopic surgical procedures including, in general, a handle, an elongated shaft, and a tip. The shaft is coupled to the handle, and the tip is pivotally coupled to the distal end of the shaft for articulation about an articulation axis. The tip includes two opposed jaws. The jaws are pivotally coupled at a pivot axis for movement between an open position and a closed position, and at least one of the jaws has a camming portion proximal to the pivot axis of the jaws. The instrument further includes a camming driver disposed in the shaft for reciprocating movement between a proximal position and a distal position. The camming driver has a camming portion. The drive camming portion contacts the jaw camming portion at a cam point. Preferably, the jaw camming portion is a hemispherically-shaped projection and the driver camming portion is a ramp. Movement of the camming driver from its proximal position to its distal position causes the jaws to pivot from their open position to their closed positions.

U.S. Pat. No. 4,313,431 to Frank, which is incorporated herein by reference, describes endoscopic apparatus with a laser light conductor used for irradiating bladder tumors in man with a laser light beam. For this purpose a jacketed light conducting fiber is arranged in parallel to an optical viewing device. A rigid bushing is secured to the output end of the light conducting fiber. The bushing includes a hinging member connected to a push rod or cable pull for remote manipulation of the output end of the light conducting fiber. The light conducting fiber is axially shiftable in its jacket. For this purpose a shifting device, located at the input or operating end of the apparatus is operatively connected to the light conducting fiber.

U.S. Pat. No. 7,201,731 to Lundquist et al., which is incorporated herein by reference, describes a medical treatment device comprising an elongate probe member having proximal and distal extremities. The elongate probe member has a longitudinal axis and at least one passageway extending from the proximal extremity to the distal extremity. A guide is mounted in the at least one passage of the elongate probe member and has proximal and distal extremities with the distal extremity of the guide being in the vicinity of the distal extremity of the elongate probe member. The guide has an opening in the distal extremity and a lumen extending from the proximal extremity to the opening in the distal extremity. A needle is slidably disposed in the lumen of the guide. The needle is in the form of a tube having an axial lumen extending therethrough. A control mechanism is coupled to the proximal extremity of the elongate probe member and is secured to the needle for advancing and retracting the needle relative to the guide.

U.S. Pat. No. 6,454,762 to Rosler et al., which is incorporated herein by reference, describes an instrument for applying light, particularly laser light, to the human or animal body. A tubular shaft is provided into which a flexible light waveguide may be inserted, where a light-emitting end of the waveguide comes to rest at a distal end portion of the tubular shaft. The distal end portion of the tubular shaft is pivotally connected with the remaining portion of the tubular shaft, so that the distal end portion may be pivoted away from the longitudinal axis of the tubular shaft. Manipulating devices are provided at the proximal end of the tubular shaft for pivoting the distal end portion. The manipulating devices include at least one movable operating element, which is directly connected to the distal end portion by means of an actuator element.

U.S. Pat. No. 5,284,474 to Adair, which is incorporated herein by reference, describes a disposable trocar for use as a gas insufflation needle, for insertion through the abdominal wall of a patient and into a body cavity. It has an outer sheath with a tubular body, a distal open end and a proximate open end. The distal end may have means which is expandable after the trocar has been inserted into the body cavity to minimize dislocation of the outer sheath during use. A cannula is removably received within the sheath which has a sharp distal end extendable beyond the distal end of the sheath and an enlarged head at the proximate end of the cannula. The head has a flat land which is engageable with the proximate end of the sheath to limit the extension of the distal end of the cannula beyond the distal end of the sheath. A rod is mounted within the cannula for longitudinal movement between a retracted position and an extended position. A blunt member is provided at the distal end of the rod extending beyond the sharp distal end of the cannula when in extended position. A spring, in a cavity in the head, is attached to the proximate end of the rod urging it toward the extended position. The method of using the trocar is also disclosed.

The following patents and patent application, which are incorporated herein by reference, may be of interest: U.S. Pat. No. 4,499,899 to Lyons, I I I; U.S. Pat. No. 5,312,399 to Hakky et al.; U.S. Pat. No. 5,320,617 to Leach; U.S. Pat. No. 5,380,321 to Yoon; U.S. Pat. No. 5,527,331 to Kresch et al.; U.S. Pat. No. 6,971,989 to Yossepowitch; U.S. Pat. No. 7,309,341 to Ortiz et al.; and US Patent Application Publication 2007-0093790 to Downey et al.

Despite the many advantages underlying the incorporation of lasers into endoscopic devices, problems of design and implementation have thus far limited the practical use of such devices. These shortcomings generally stem from the way the laser light is delivered to tissues. In the majority of laser resectoscopes, the light emanates from the tip of the fiber optic, either along its longitudinal axis, or in some cases (e.g. the devices disclosed in U.S. Pat. Nos. 5,416,878; 5,487,740; 5,647,867; and 6,802,838) perpendicular to the axis of the fiber optic by means of additional optics. Even designs in which the light emanates from the fiber optic at a relative angle to its longitudinal axis do not allow for optimal accuracy in positioning of the light beam, since manipulation of the fiber optic, and hence of the direction of the emitted light, is limited to linear motion along its axis via a manual control mechanism located at the endoscope's proximal end. The use of such a manual control mechanism limits the accuracy and reproducibility of the placement of the emitted laser beam. Another concern derives from the use of an intense beam of light focused onto a small spot (inherent properties of laser light). Overexposure of the tissue to be resected to high laser intensity can lead to damage of the underlying tissues, to the point of unwarranted tissue perforation. Furthermore, uncontrolled rotation of the fiber optic results in the laser beam being emitted in several directions, not just in the direction of the tissue to be resected. Should the laser beam intersect the medical instrument itself, it is likely that the instrument will suffer serious damage. These safety concerns have also restricted the use of laser resectoscopes, despite the advantages outlined above.

Thus, there remains a long-felt need for a working tool that enables more accurate manipulation of the emitted laser light beam both rotationally around the axis of the fiber and horizontally relative to the axis of the working tool. Because uncontrolled rotation of the emitted light may damage the fiber optic cable through which the operation is viewed, the need is thus not only for a laser working tool that enables manipulation of the emitted light rotationally around the axis of the fiber optic, but also for one that permits rotation through a user-controllable angle of no more than 360°.

In order to be maximally useful in the operating room and to be compatible with the existing designs with which users are familiar, the controls at the proximal end of the working tool need to be designed to provide linear rather than rotational motion. There is thus a need not just for a laser working tool that provides rotational motion of the emitted light, but one that enables more accurate positioning of the emitted laser beam via incorporation of a mechanical rather than manual means of effecting the translation and rotation of the fiber optic. In addition, there remains a long-felt need for providing the operator with a means for converting linear motion at the proximal end (easily controlled by the user) to rotational motion at the distal end.

Furthermore, there remains a long-felt need for a design for a laser working tool that can be incorporated into an endoscopic medical tool that obviates the safety concerns outlined above, namely, the high probability of inadvertent damage to healthy tissue or to the endoscope itself.

All patents, patent applications, and other references that are cited in the present patent application are incorporated herein by reference.

### SUMMARY OF THE INVENTION

An embodiment of the invention provides a working tool for handling and accurately actuating a side-firing fiber optic waveguide (SiFOW). The SiFOW's firing distal end extends from the distal portion of the working tool, previously inserted through its proximal portion. The SiFOW has a light transmitting distal end and light receiving proximal end connected to a laser light source external to the working tool. The SiFOW is utilized for treating a targeted tissue located within a body cavity of a patient, especially by means of light beam facilitated vaporization/ablation and coagulation of the tissue. The working tool has a distal portion (an elongated shaft) insertably located adjacent to the tissue to be treated within the body of a patient; and a proximal portion (a handle assembly, in connection with the shaft) located out of the body, held and operated by a surgeon. The SiFOW is positioned by being reversibly introduced into a designated pathway and/or is reversibly held in position by being grasped at least at one point along the shaft; hence the shaft facilitates the SiFOW's accurate actuation. The accurate actuation is provided by a working tool's actuating mechanism, comprising a handle assembly further which comprises at least one handle, translating the handle's linear movement to SiFOW's self-limited rotation; and optionally, at least one knob providing SiFOW's self-limited adjustable linear positioning; and a quick connector.

An embodiment of the invention provides the working tool as defined above, comprising two independently-operated, self-limited (a) rotational, and (b), linear SiFOW actuating mechanisms.

An embodiment of the invention provides the working tool as defined above, wherein the self-limited rotational SiFOW actuating mechanism is selected from a group of mechanisms consisting of (i) rotating screw and fixed nut; (ii) rotating nut and fixed screw; (iii) a spiral track and traveling pin, or any other means of producing rotational motion.

An embodiment of the invention provides disclose the working tool as defined above, wherein the maximum possible rotation angle φₘₐₓ around the longitudinal axis of the shaft is limited to φₘₐₓ absolute values of about 20° to about 280°, preferably φₘₐₓ absolute values of about 30° to about 60°.

An embodiment of the invention provides the working tool as defined above, wherein the self-limited linear SiFOW actuating mechanism is selected from a group of mechanisms consisting of (i) spur gear mechanism; (ii) conjugated screws rotating mechanism and/or spiral tracks mechanisms, (iii) a sliding lock plate which pushes the rotational mechanism in a linear direction; (iv) a straight track and traveling pin; or any other means of producing linear motion.

An embodiment of the invention provides the working tool as defined above, wherein the self-limited linear SiFOW actuating mechanism incorporates a position temporary locking mechanism selected from a group of mechanisms consisting of (i) a friction mechanism, (ii) an incremental toothed mechanism; or any other means of producing linear motion.

An embodiment of the invention provides the working tool as defined above, wherein the described mechanism enables the surgeon to temporarily pull the SiFOW proximally out of view, thus obtaining a clear undisturbed view of the treated tissue. The SiFOW automatically returns to its distal position, when let free by the surgeon.

An embodiment of the invention provides the working tool as defined above, wherein the maximum possible reciprocally-provided linear movement RLMₘₐₓ along the longitudinal axis of the shaft is limited to RLMₘₐₓ absolute values of about 0 mm to about 50 mm, preferably to absolute values of about 1 to about 25 mm.

An embodiment of the invention provides the working tool as defined above, wherein the rotation mechanism actuates the SiFOW by means of a hollow twisted rectangle (high pitch screw) and a complimentary nut. The screw is fixed linearly while free to rotate around its longitudinal axis, and the nut is actuated linearly along the screw by the distal handle, thus causing the screw to rotate accordingly.

An embodiment of the invention provides the working tool as defined above, wherein the linear mechanism actuates the SiFOW by means of a spur gear and toothed rack mechanism comprising (a) a circular hollow tooth rack (CHTR) in mechanical connection with the high pitch screw, while the SiFOW is reversibly inserted through both (b) a reciprocal motion knob (RMK), which is connected to and rotates the spur gear, the spur gear is mechanically coupled with the CHTR and activates it linearly; (c) a position holding friction mechanism that temporarily friction locks the spur gear; and (d) a shell housing adapted to provide mechanical support and bearing for the CHTR, spur gear, knob, friction mechanism and mechanisms thereof.

An embodiment of the invention provides the working tool as defined above, further comprising a motor, gear and cam shaft mechanism facilitated for rotating, and/or a linear reciprocating means for actuating the SiFOW. It is acknowledged in this respect that even though the motor in the drawn embodiment is designed only for rotating the SiFOW, a motor for linearly actuating the SiFOW may be easily added or replace the drawn one.

An embodiment of the invention provides the working tool as defined above, wherein the MGRL is selected from a group consisting of: direct or indirect drive, electric, pneumatic, hydraulic, piezoelectric, electro magnetic, ultra sonic, galvanic, bimetallic, Peltier engine or any combination thereof.

An embodiment of the invention provides the working tool as defined above, wherein the motor is operated by automatic means, semi automatic means or manual means; and optionally wherein the operating speed is controlled by the surgeon; the means selected from a group preferably consisting of handle, pedal, voice command, computer mediated means, robotic interface, or any combination thereof.

An embodiment of the invention provides the working tool as defined above, wherein the emitting tip of the SiFOW is either focused or defocused in a predetermined manner; the defocusing is preferably provided by means selected from a group consisting of: mechanical vibration, ultrasonic, acoustic, piezoelectric, or other reciprocating motion.

An embodiment of the invention provides a method for treating a targeted tissue located within a body cavity of a patient, especially by means of light beam facilitating vaporising/ablating and coagulating of the tissue, by a means of a laser working tool. The method comprising steps of (a) obtaining a working tool for handling and accurately actuating a side-firing fiber optic waveguide (SiFOW); the working tool having a distal portion (an elongated shaft) insertably located adjacent to the tissue to be treated within the body of a patient; and a proximal portion (handle, in connection with the shaft) located out of the body, held and operated by a surgeon (b) providing the working tool with an actuating mechanism, namely (i) providing at least one handle with a linear-to-rotational translating means; and optionally (ii), providing at least one knob adapted for enabling the SiFOW's to move linearly; (c) inserting the SiFOW from the proximal portion of the working tool through its distal portion, preferably by introducing the SiFOW into a designated pathway within the actuating mechanism and/or shaft, and/or at least reversibly holding the SiFOW in position by grasping it at one or more points along the shaft, thus facilitating the SiFOW's accurate actuation; (d) connecting a laser light source external to the working tool to the SiFOW's proximal end; (e) providing the SiFOW's with means for accurate rotational actuation by squeezing the handle and thus translating linear motion of the handle to rotational movement of the SiFOW with respect to the axis of the shaft; (f) optionally, providing the SiFOW's with means for accurate linear actuation, by operating the linear actuation operating knob; and (g) transmitting laser light from a light outlet located adjacent to the distal end of the SiFOW, emitting light until the treatment of the area is complete.

An embodiment of the invention provides the method as defined above, wherein step (e) is provided by facilitating a motor and gear-facilitated rotating and/or a linear reciprocating (MGRL) means for actuating the SiFOW.

An embodiment of the invention provides the method as defined above, wherein the method further comprises a step of selecting the MGRL from a group consisting of direct or indirect drive, electric, pneumatic, hydraulic, piezoelectric, electromagnetic, ultra sonic, galvanic, bi-metallic, Peltier drive or any combination thereof.

An embodiment of the invention provides the method as defined above, wherein the method comprises the steps of operating the motor by automatic means, semi automatic means or manual means; and optionally wherein the operating speed is controlled by the surgeon controlled means; the means selected from a group preferably consisting of handle, pedal, voice command, computer mediated means, robotic interface, or any combination thereof.

An embodiment of the invention provides the method as defined above, further comprising a step of providing the working tool with linear actuating motor; alternatively, said method comprising a step of providing the working tool with a linear actuating motor and not a rotating actuating motor.

An embodiment of the invention provides the method as defined above, wherein the method further comprises focusing or defocusing the emitting tip in a predetermined manner; the defocusing is preferably provided by selecting means from a group consisting of mechanical vibration, ultrasonic, acoustic, piezoelectric, or otherwise reciprocating motion.

An embodiment of the present invention provides apparatus, including:
an intraluminal tube having a proximal end and a distal end, the tube being shaped to provide a longitudinal lumen;
an optical unit in communication with the tube and configured illuminating and viewing a view distal to the distal end of the tube;
a handle assembly coupled to the proximal end of the tube;
a treatment element configured to be disposed within the lumen of the tube, the treatment element having a proximal end and a distal end, and the treatment element having a resting position thereof in which the distal end of the treatment element extends distally from the distal end of the tube and is in the view distal to the distal end of the tube;
a spring mechanism coupled to the handle assembly; and
a user-force receiver coupled to the spring mechanism and to the treatment element, and configured to be:
   displaced, with respect to the handle assembly, by a user of the apparatus, and by being displaced to: (a) apply a force to the spring mechanism, and (b) pull the distal end of the treatment element proximally, out of the view, irrespectively of a rotational position of the treatment element, and
   released, and by being released, to: (a) be displaced in response to a force applied to the user-force receiver by the spring mechanism, and (b), return the treatment element to the resting position thereof.

In an embodiment, in the resting position, the distal end of the treatment element is configured to be disposed up to 25 millimeters distal to the distal end of the tube.

In an embodiment, the treatment element includes a side-firing fiber optic waveguide (SiFOW) configured to treat tissue of a patient.

In an embodiment, the treatment element is configured to be slidably advanced through the tube from the proximal end of the tube toward the distal end of the tube.

In an embodiment, the treatment element is coupled to the user-force receiver, and, in response to the force applied to the spring mechanism by displacing the user-force receiver, the treatment element is pulled proximally with respect to the tube.

In an embodiment, the spring mechanism is coupled to the user-force receiver, and the user-force receiver is coupled to a portion of the treatment element, and, in response to displacement of the user-force receiver, the treatment element is pulled proximally with respect to the tube and force is applied to the spring mechanism.

In an embodiment, the apparatus includes a motor configured to control longitudinal motion of the treatment element.

In an embodiment, the apparatus includes a motor configured to control rotational motion of the treatment element.

In an embodiment, the motor is configured to control longitudinal motion of the treatment element.

In an embodiment, the handle assembly includes a user-grasping member configured to be longitudinally displaceable with respect to the tube.

In an embodiment, the treatment element is configured to be rotated about a longitudinal axis of the tube in response to longitudinal displacement of the user-grasping member.

In an embodiment, the treatment element is configured to be rotated no more than 360 degrees about a longitudinal axis thereof, in response to a full longitudinal displacement of the user-grasping member.

In an embodiment, the treatment element is configured to be rotated between 20 degrees and 280 degrees about the longitudinal axis thereof, in response to the full longitudinal displacement of the user-grasping member.

In an embodiment, the treatment element is configured to be rotated between 20 degrees and 80 degrees about the longitudinal axis thereof, in response to the full longitudinal displacement of the user-grasping member.

In an embodiment, the method includes:
a nut coupled to the user-grasping member, and
a screw locked linearly while free to rotate with respect to the nut, the screw being configured to be threaded through the nut, and shaped to define a longitudinal lumen for passage therethrough of the treatment element, and, in response to longitudinal displacement of the user-grasping member:
the nut is configured to move longitudinally with respect to the screw and facilitate rotation thereof, and
the treatment element is configured to be rotated responsively to the rotation of the screw.

In an embodiment, a portion of the treatment element is coupled to the user-force receiver, and the user force receiver is coupled to the spring mechanism which is coupled to the proximal end of the screw in a manner in which the spring mechanism and the screw are locked rotationally. The spring mechanism, the user force receiver, and the treatment element may travel linearly, while the screw is unable to travel linearly. In response to the force applied to the user-force receiver, the user-force receiver is displaced proximally, pulling with it the treatment element and the spring mechanism proximally with respect to the tube.

In an embodiment, the screw is shaped to define a helical thread having a pitch, the pitch determining an extent of rotation of the treatment element with respect to movement of the nut.

An embodiment of the present invention provides apparatus, including:

an intraluminal tube having a proximal end and a distal end, the tube being shaped to provide a longitudinal lumen;
a treatment element having a proximal end and a distal end, the treatment element configured to be disposed within the lumen of the tube;
a handle assembly coupled to the proximal end of the treatment element; and
a user offset controller coupled to the handle assembly, and configured to set an offset of the distal end of the treatment element with respect to the distal end of the tube.

In an embodiment, the offset controller is configured to set the offset of the distal end of the treatment element irrespectively of a rotational position of the treatment element.

In an embodiment, the distal end of the treatment element is configured to be disposed up to 25 millimeters distal to the distal end of the tube.

In an embodiment, the treatment element includes a side-firing fiber optic waveguide (SiFOW) configured to treat tissue of a patient.

In an embodiment, a portion of the treatment element is coupled to the offset controller, and, in response to the force applied to the offset controller, a position of the treatment element is offset with respect to the tube.

In an embodiment, the treatment element is configured to be slidably advanced through the tube.

In an embodiment, the apparatus includes a motor configured to control longitudinal motion of the treatment element.

In an embodiment, the apparatus includes a motor configured to control rotational motion of the treatment element.

In an embodiment, the motor is configured to control longitudinal motion of the treatment element.

In an embodiment, the user offset controller includes:
a first gear, coupled to a portion of the treatment element;
a rotatable knob; and
a second gear, coupled to the knob and in communication with the first gear,
and the first gear is displaced longitudinally with respect to the second gear in response to rotation of the knob.

In an embodiment, a rotational axis of the first gear is substantially perpendicular to a rotational axis of the second gear.

In an embodiment, the offset controller is configured to offset a longitudinal position of the treatment element by longitudinally displacing with respect to the tube the first gear and the portion of the treatment element coupled thereto, in response to rotation of the knob.

In an embodiment, the handle assembly includes a user-grasping member configured to be longitudinally displaceable with respect to the tube.

In an embodiment, the treatment element is configured to be rotated about a longitudinal axis of the tube in response to longitudinal displacement of the user-grasping member.

In an embodiment, the offset controller is configured to set the offset of the distal end of the treatment element irrespectively of a rotational position of the treatment element.

In an embodiment, the treatment element is configured to be rotated no more than 360 degrees about a longitudinal axis thereof, in response to a full longitudinal displacement of the user-grasping member.

In an embodiment, the treatment element is configured to be rotated between 20 degrees and 280 degrees about the longitudinal axis thereof in response to the full longitudinal displacement of the user-grasping member.

In an embodiment, the treatment element is configured to be rotated between 20 degrees and 80 degrees about the longitudinal axis thereof in response to the full longitudinal displacement of the user-grasping member.

In an embodiment, the apparatus includes:
a nut coupled to the user-grasping member, and
a screw locked linearly while free to rotate with respect to the nut, the screw being configured to be threaded through the nut, and shaped to define a longitudinal lumen for passage therethrough of the treatment element,
and, in response to longitudinal displacement of the user-grasping member:
the nut is configured to move longitudinally with respect to the screw and facilitate rotation thereof, and
the treatment element is configured to be rotated responsively to the rotation of the screw.

In an embodiment, the screw is shaped to define a helical thread having a pitch, the pitch determining an extent of rotation of the treatment element with respect to movement of the nut.

In an embodiment, a portion of the treatment element is coupled to the offset controller, and, in response to the force applied to the offset controller, the offset controller is displaced, and responsively, the treatment element is displaced, such that the longitudinal position of the treatment element is displaced within the screw and is offset with respect to the tube.

In an embodiment, the user offset controller includes:
a first gear, coupled to a portion of the treatment element and coupled to the screw in a fashion such that they are locked rotationally while the first gear is free to travel linearly with respect to the screw;
a rotatable knob; and
a second gear, coupled to the knob and in communication with the first gear,
and the first gear is displaced longitudinally with respect to the second gear in response to rotation of the knob.

In an embodiment, a rotational axis of the first gear is substantially perpendicular to a rotational axis of the second gear.

In an embodiment, the portion of the treatment element is disposed within the first gear of the offset controller, and the offset controller is configured to offset a longitudinal position of the treatment element by longitudinally displacing with respect to the tube the first gear and the treatment element disposed within the first gear, in response to the rotation of the knob.

An embodiment of the present invention provides a method, including:
advancing through a lumen a tube having a proximal end and a distal end, the tube being configured to be coupled to a treatment element having a proximal end and a distal end, and the treatment element having a resting position thereof in which the distal end of the treatment element extends distally from the distal end of the tube;
viewing a view distal to the distal end of the tube;
irrespectively of a rotational position of the treatment element, retracting the distal end of the treatment element to a site proximal to the view distal to the distal end of the tube, while applying a force to a spring mechanism in communication with the treatment element by pulling a force receiver coupled to the spring mechanism; and
returning the treatment element to the resting position thereof by releasing the force receiver.

In an embodiment, the method includes advancing through the tube the treatment element to the resting position thereof, and placing the distal end of the treatment element distally to the distal end of the tube and in the view distal to the distal end of the tube following the advancing.

In an embodiment, advancing the treatment element through the tube includes advancing the treatment element through the tube until a distal end of the treatment element is disposed up to 25 mm distal to the distal end of the tube.

In an embodiment, the method includes:
advancing through a lumen a tube having a proximal end and a distal end, the tube being configured to be coupled to a treatment element having a proximal end and a distal end; and
offsetting a position of the treatment element from a first position to a second position with respect to the distal end of the tube by manipulating an offset controller that secures the treatment element at the second position.

In an embodiment, offsetting the position of the treatment element includes offsetting the position of the treatment element irrespectively of a rotational position of the treatment element.

In an embodiment, the method includes advancing through the tube the treatment element to the first position, and placing the distal end of the treatment element distally to the distal end of the tube and in the view distal to the distal end of the treatment element following the advancing.

In an embodiment, advancing the treatment element through the tube includes advancing the treatment element through the tube until a distal end of the treatment element is disposed up to 25 mm distal to the distal end of the tube.

### BRIEF DESCRIPTION OF THE DRAWINGS

In order to better understand the invention and its implementation in practice, a plurality of embodiments will now be described, by way of non-limiting example only, with reference to the accompanying drawings, in which

FIGS. 1A-1B illustrate a first embodiment of a working tool (100) of the present invention; FIG. 1A discloses a side view of an embodiment including, inter alia, the laser handle, the SiFOW, a system for insertion and continuous irrigation (outer sheath) and a scope;

FIG. 1B discloses a side view of the working tool 100 as is, i.e., when connected only to the SiFOW;

FIG. 2A-2B present a side view and a perspective view (side and rear view), respectively, of the liner-to rotational actuator which consists of a travelling nut 22 and a linearly fixed high-pitched screw 20;

FIG. 3 presents a perspective view (side and rear view) of the interior portion of sleeve-like element 26 (cf. 26 in FIG. 2B);

FIG. 4A presents a detailed perspective view of travelling nut 22 & a high-pitched screw 20 liner-to-rotational translation mechanism 110; FIGS. 4B and 4C present front and rear perspective views of the sleeve-like element 26;

FIGS. 5A-5B illustrate schematic side-views of another embodiment (namely working tool 200) which is adapted to handle and accurately actuate SiFOW 1 rotationally and or linearly;

FIGS. 6A-6C illustrate side view and two perspective views in a schematic side view, side and rear view and side and frontal view of device 200, respectively;

FIG. 7 presents a perspective view of the independent (i) nut and high-pitch screw rotational mechanism and (ii) the linear-actuating mechanism of working tool 200;

FIGS. 8A and 8B illustrate side views of a motorized version (300) of the aforesaid working tool, adapted for a motor, gear and cam shaft mechanism facilitated for rotating the SiFOW, and a linear actuating mechanism of the SiFOW;

FIGS. 9A-9C illustrate side view and two perspective views of mechanism 40;

FIG. 10 presents a perspective view of the independently operated (i) motor-facilitated, accurate and self-limited rotating mechanism (40), and (ii), manual linear mechanism coupled to it; and,

FIG. 11A presents a perspective view elongated element 26 according to one embodiment of the invention; FIGS. 11B and 11C present perspective views of an SiFOW-connector 12, which is designed such that the insertion of SiFOW 1 to the connector is performed via a designated slot.

### DETAILED DESCRIPTION OF EMBODIMENTS OF THE INVENTION

The following description is provided so as to enable any person skilled in the art to make use of the present invention and sets forth the best modes contemplated by the inventors of carrying out the invention. It will be apparent to one skilled in the art, however, that there are several embodiments of the invention that differ in minor details of construction without affecting the essential nature thereof, and therefore the invention is not limited by that which is illustrated in the figures and described in the specification.

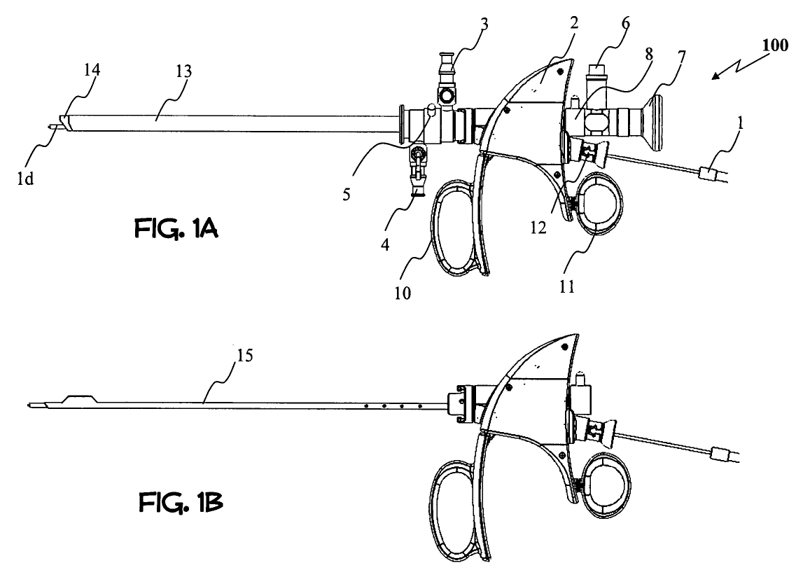

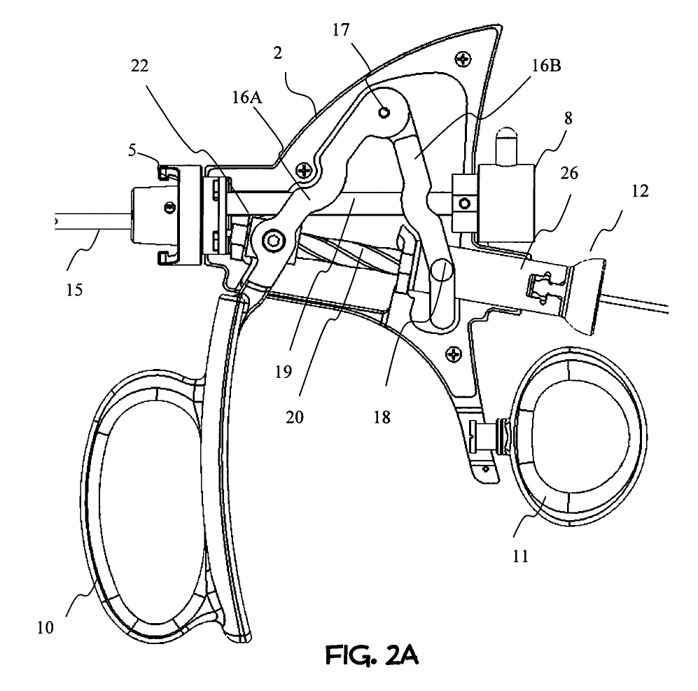

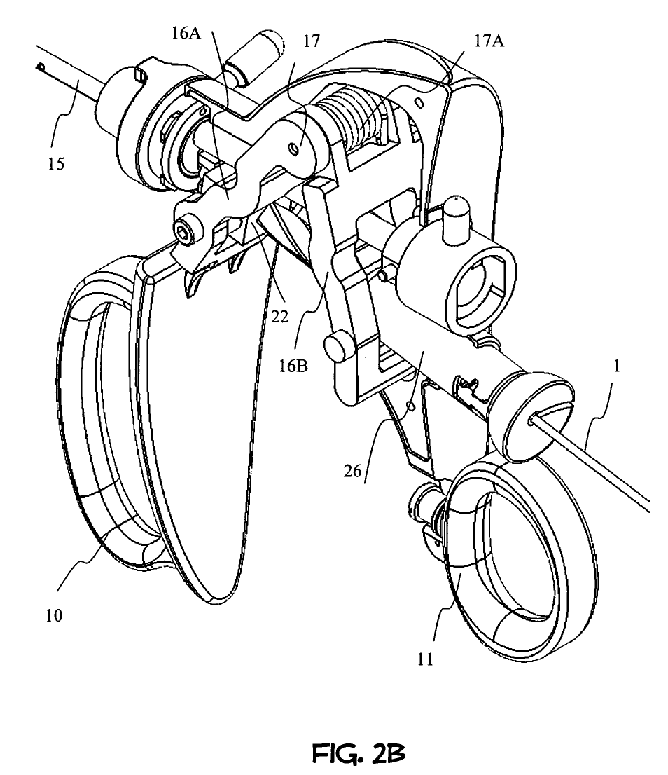

Reference is now made to FIGS. 1A-1B, illustrating in a schematic manner one embodiment of a working tool (100) of the present invention, especially adapted to treat a targeted tissue located within a body cavity of a patient, preferably by means of light beam facilitated vaporization/ablation and coagulation of the tissue. FIG. 1A discloses a side view of an embodiment of a laser handle assembly coupled to a set of sheaths (inner and outer) utilized as a working channel and an irrigation system and additionally showing the proximal end of a scope. Fluid (normally a saline solution) enters via inlet port 3 into inner sheath 14 (flowing distally), while irrigating and inflating the body cavity, and exits (flowing proximally) through outer sheath 13 via exit port 4. In order to enable the operator to view the operation of the working tool, an optical unit, typically comprising an optical fiber, is inserted within the system, attached to the proximal end of the working tool by means of connector (8), comprising an eyepiece (7) for viewing the procedure occurring at the distal end, inlet (6) positioned on the optical system is utilized for connecting an external light source to illuminate the distal end. The handle assembly of the working tool usually includes two handles, or user-grasping members. In the embodiment illustrated in FIGS. 1A and 1B, at least one of the handles comprises (a) a movable spring-loaded active handle (10) configured to be displaced longitudinally with respect to sheaths 13 and 14 and tube 15, which handle is attached to the body of the working tool via an axis (not shown) and a spring (not shown); and optionally, (b) a second stationary passive handle (11). Typically, the handle assembly is coupled to a proximal end of tube 15. It is yet well in the scope of the invention wherein the rear handle is active and front handle is passive. The actuator, described below, is held within a cover (2), which comprises for example two shelling pieces, e.g., a left and right covers. A treatment element, e.g., a side-firing fiber optic waveguide (SiFOW, 1), passes through the actuators and through tube (15) (i.e., from a proximal end of tube 15 toward a distal end of tube 15), which is itself located entirely within the outer sheath 13 and inner sheath 14. The distal tip (Id) of the SiFOW extends beyond the end of tube 15, and the sheaths 13 and 14, such that light beam facilitated vaporization/ablation and coagulation of tissue will not interfere with the operation of the working tool itself. A portion of the SiFOW is interconnected to working tool 100 by connector 12. Typically, a portion of the SiFOW is rigidly coupled to connector 12. For embodiments in which the SiFOW is passed through working tool 100 from a proximal end of the handle assembly toward a distal end of tube 15, the SiFOW is passed through a lumen of connector 12, through the working tool, and toward the distal end of tube 15, until the distal end of the SiFOW is disposed distal to the distal end of tube 15. Once the distal end of the SiFOW is disposed distal to the distal end of tube 15, connector 12 locks into part 26 (See FIG. 2B) thus the SiFOW is locked in place. In some embodiments, the SiFOW is surrounded by a connector 12 which is rigidly positioned at a predetermined distance and angle from the distal end of the SiFOW. The lock is configured to be coupled to connector 12, thereby locking the SiFOW in place and preventing the SiFOW from sliding distally beyond a predetermined distance. For example, the SiFOW is slid distally until the distal end of the SiFOW is disposed up to 25 mm distal to the distal end of tube 15, and the lock locks the SiFOW in place, thereby inhibiting continued distal sliding of the SiFOW beyond 25 mm from the distal end of tube 15. Additionally, outer sheath 13 and inner sheath 14 are interconnected with working tool 100 by connector 5. In the embodiment illustrated in FIG. 1A, the distal potion of the inner sheath 14 protrudes from outer sheath 13. FIG. 1B discloses a side view of the working tool 100 as is, i.e., when connected only to the SiFOW, without the sheaths and optical system. As shown in FIG. 1A, the SiFOW has a longitudinal resting position thereof in which a distal end of the SiFOW is disposed distal to the distal end of tube 15 and the distal ends of sheaths 13 and 14.

Reference is now made to FIGS. 2A-2B, presenting the liner-to rotational actuator which consists of a travelling nut 22 and a high-pitched screw 20. Typically, nut 22 is coupled to handle 10. The screw (e.g., a twisted rectangle) is threaded through the nut. A bore through the centre of the screw along its longitudinal axis allows SiFOW 1 to pass freely through. Moving or squeezing the active handle (here front handle 10) causes the travelling nut 22 to move along screw 20, causing screw 20 to turn; thus the actuator converts linear motion of the handle into rotational motion of SiFOW 1 rotationally coupled through sleeve 26 as later described. In one alternative embodiment, in place of the rotational actuator, a linear motion mechanism is provided such that linear motion of the handles is converted into linear motion of the SiFOW along its axis (not shown). A further alternative embodiment comprises a dual-action actuator that consists of both (i) rotational and (ii) linear actuators, and a switch that enables the user to choose between linear-to-rotational and linear-to-linear actuation for any given motion of the active handle (not shown).

It is in the scope of the invention for the actuator to be angularly limited (self-limiting). That is, the total motion of the distal end of the SiFOW is limited by the actuator itself, as the SiFOW can only rotate within the limits set by the linear to rotational mechanism actuated by the active handle. The rotational motion of the SiFOW, the angle through which the tip of the SiFOW (and hence the emitted laser beam) can travel, i.e., rotate about a longitudinal axis thereof, is normally limited to no more than 360 degrees, e.g., between 20 degrees and 280 degrees, typically between 20 degrees and 80 degrees by way of illustration and not limitation. That is, the SiFOW rotates no more than 360 degrees during a full longitudinal displacement of active handle 10 proximally toward passive handle 11, and typically rotates no more than 80 degrees during such a displacement. The angle of rotation can be set by choice of pitch (of the screw) verses travel (of the nut); the lower the pitch of the screw, the larger the angle of rotation through which motion of the active handle from its most distal to its most proximal position will drive the rotation of the SiFOW.

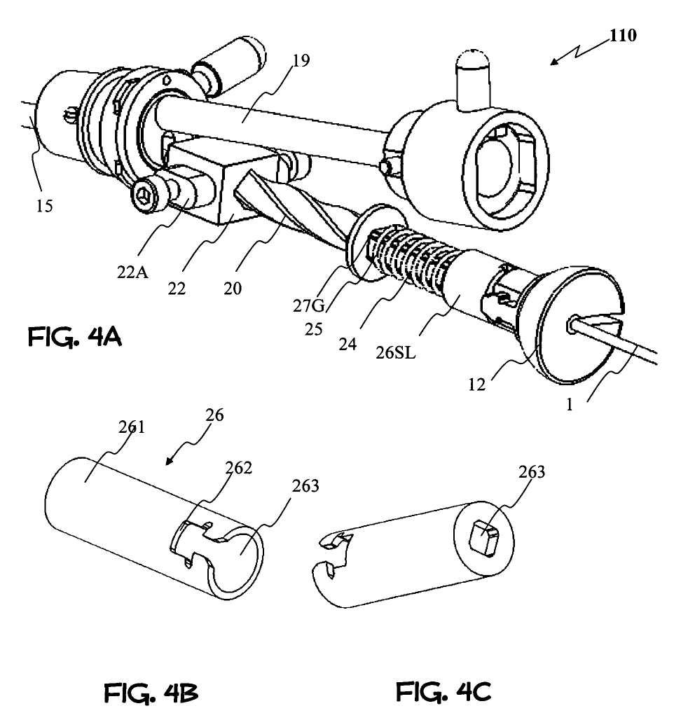

Reference is still made to FIGS. 2A-2B, presenting a general view of a travelling nut 22 & a high-pitched screw 20 liner-to-rotational translation mechanism 110 (See FIG. 4A). Working tool 100 comprises, in a non-limiting manner, optic truck (19), comprising an optical fiber, in connection to an eyepiece, CCD or video by connector 8; SiFOW connector (see members 26 and 12); passive rear handle 11; active front handle 10; tube 15, outer sheath connector 5, and housing 2. Handle 10 is mechanically connected to pivot 17 via lever 16A & 16B, wherein the pivot is spring (17A) activated, and wherein pivot 17 is also affixed to housing 2 via pivot 18. The external view of sleeve-like element 26 is also presented.

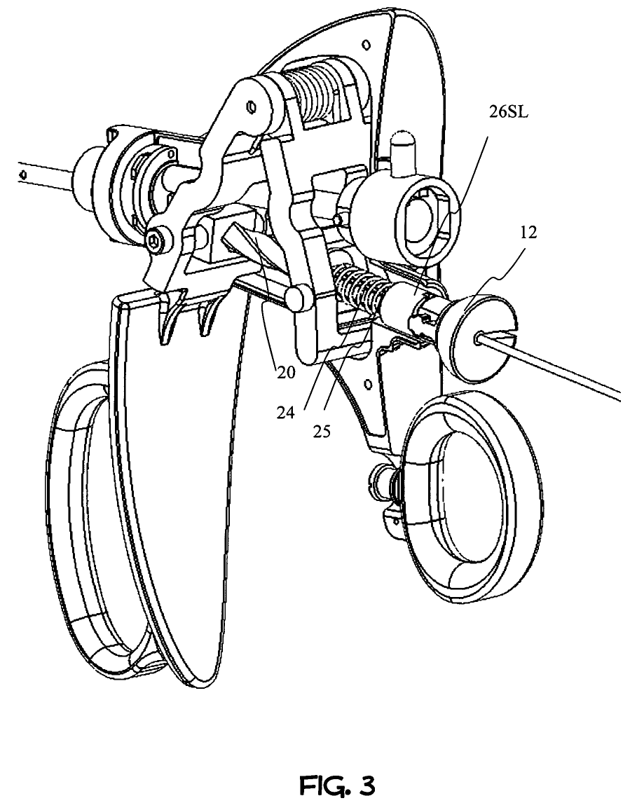

Reference is now made to FIG. 3, presenting the interior portion of sleeve-like element 26 (not shown), which is an envelope with a distal polygonal (e.g., rectangular) distal hole, and a proximal SiFOW connector. The inner portion of sleeve-like element 26 contains a polygonal (e.g., rectangular) extension 25 of screw 20, enveloped by a spring mechanism comprising at least one spring 24, and a spring locker 26SL.

Typically, the optical fiber of optic trunk 19 extends toward the distal ends of sheaths 13 and 14 and tube 15 and is configured to view a view distal to the distal ends of tube 15 and of sheaths 13 and 14. It is well in the scope of the invention, for the described mechanism to enable the surgeon to temporarily pull the SiFOW proximally out of view, i.e., by displacing the SiFOW from its longitudinal resting position by pulling the distal end of the SiFOW out of the view, thus obtaining a clear undisturbed view of the treated tissue, the proximal movement of sleeve 26 and connector 12, and causing spring 24 to shorten/load, thus returning the SiFOW to its distal position when let free/loose. Typically, a portion of the SiFOW is coupled to spring 24 by being passed therethrough. The SiFOW then passes through a lumen of screw 20. Typically, spring 24 is indirectly coupled to a portion of screw 20.

Typically, in order to effect the movement of the spring, a user displaces a user-force receiver, e.g., the structure of connector 12, sleeve 26, or spring locker 26SL. When the force receiver is displaced with respect to the handle assembly, e.g., pulled, by a user of the apparatus, the force receiver applies a force to spring 24 and pulls the distal end of the SiFOW proximally, out of the view, irrespectively of a rotational position of the SiFOW. For example, when active handle 10 is partially or fully compressed and, responsively thereto, the SiFOW is rotated to a given rotation position (e.g., 30 degrees from its rotational resting position), the spring mechanism is able to pull the SiFOW in that rotational position, without requiring the SiFOW to first be rotated back to the rotational resting position. Since a portion of the SiFOW is rigidly coupled to connector 12, pulling on the user-force receiver applies a force to connector 12 which pulls connector 12 proximally, which in turn, applies a force to the SiFOW and pulls the SiFOW proximally. The force applied to the spring pulls linear stop groove 27G (See FIG. 4A) proximally, thereby compressing spring 24, thereby pulling on the SiFOW disposed in the screw 20. When the force receiver is released by the user of the apparatus, the force receiver is displaced in response to a force applied to the force receiver by spring 24. That is, when the spring is allowed to relax, it (a) pulls the force receiver distally, and (b) returns the SiFOW to the resting position thereof by applying a distal pushing force to stop groove 27G which, in turn, applies a distal pushing force to the SiFOW disposed therein.

Reference is now made to FIG. 4A, presenting a detailed view of a travelling nut 22 & a high-pitched screw 20 liner-to-rotational translation mechanism 110. Mechanism 110 comprises, in a non-limiting manner, travelling nut 22, here with two handle-connectors 22A; high-pitched screw 20; linear stopper groove 27G, immobilized or clasped to housing 2 (See FIG. 2A), hence providing rotation of the shaft while disenabling its reciprocal linear movement; a circular locking polygonal extension (25), connected to screw 20; a spring (24) enveloping the same; spring locker (26SL), optionally a locker screwed to the polygonal extension. According to one embodiment, mechanism 110 is in connection with optical system 19 and distal tube 15. Mechanism 110 also contains an inner pathway to SiFOW 1, and connects to the SiFOW's connector 12.

Sleeve-like element 26, which is not presented in FIG. 4A, is well defined in FIGS. 4B and 4C. Elongated element 26 consists, inter alia, of a circular external envelope 261, with polygonal (here e.g., rectangular) inner distal hole 263 and tubular inner cross-section 263, with a slotted pattern, here a female slot 262.

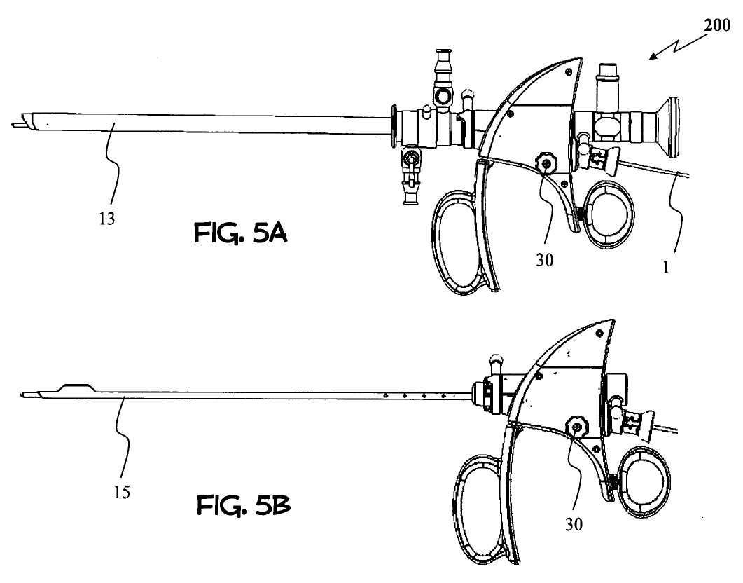

Reference is now made to FIGS. 5A-5B, illustrating schematic side-views of another embodiment (working tool 200) adapted to handle and accurately actuate a SiFOW (1). The working tool is illustrated in its connected configuration (FIG. 5A) (connected e.g., to outer sheath 13, inner sheath 14, scope and SiFOW); and in its SiFOW connected configuration (FIG. 5B). The figures also illustrate reciprocal motion knob 30.

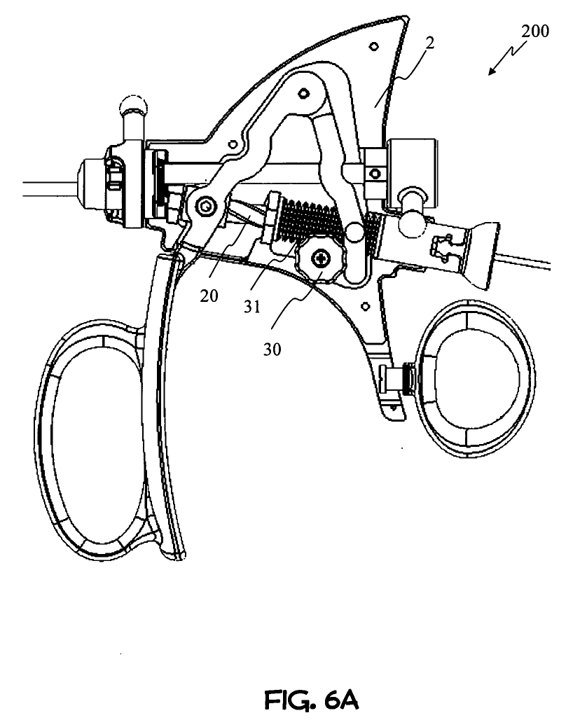

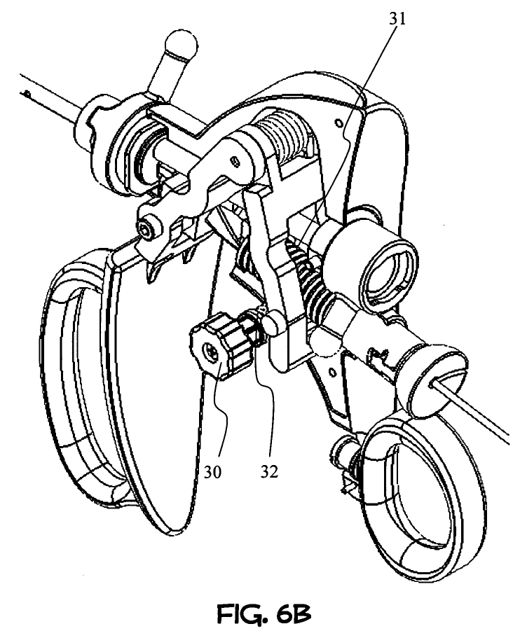

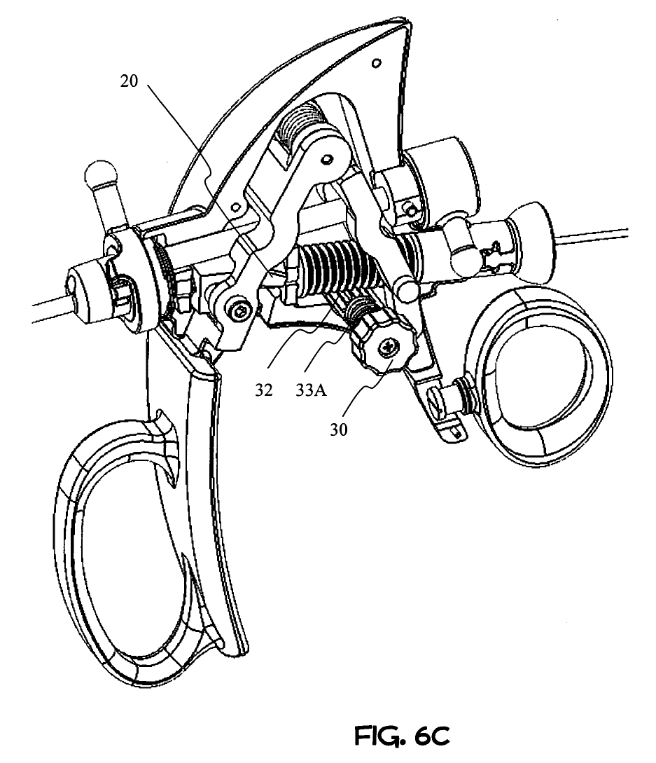

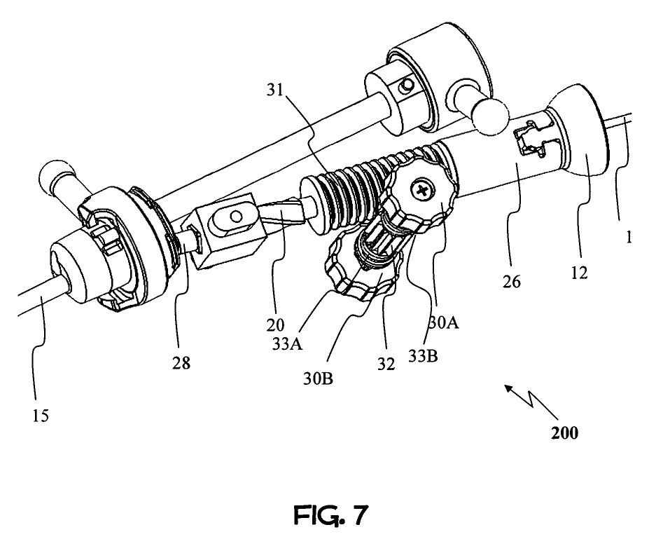

Reference is now made to FIGS. 6A-6C, illustrating in a schematic side view, side and rear view and side and frontal view of device 200, respectively, where the left portion of housing 2 is uncovered. Both linear and rotational SiFOW-actuating mechanisms are disclosed in a non-limiting manner. The rotational actuating mechanism is similar to the one described and defined before, and comprises e.g., a travelling nut & a high-pitched screw linear-to-rotational mechanism. The SiFOW linear-actuating mechanism, which in some embodiments serves as a user offset controller, comprises at least one reciprocal motion knob (also referred to as RMK, 30), provided e.g. on one side of the working tool 200, or at both opposite sides of the device; a circular hollow tooth rack (also referred to as CHTR, 31) in mechanical connection with the high pitch screw 20, a spur gear (32) with its rotational axis positioned in a perpendicular manner with respect to the rotational axis of the circular hollow tooth rack 31. Springs 33A and 33B (see FIG. 7) thrust spur gear 32 from both sides against/towards the interior walls of envelope 2, via washers; the role of the springs is, e.g., to effectively create friction and lock spur gear 32 thus affixing linearly the CHTR holding the SiFOW's tip at its last position.

Typically, a first portion of the SiFOW is coupled to CHTR 31 by being passed therethrough. The SiFOW then passes through a lumen of screw 20. Typically, CHTR 31 is indirectly coupled to a portion of screw 20. A second portion of the SiFOW is coupled to connector 12, as described hereinabove.

Reference is now made to FIG. 7, presenting the linear-actuating mechanism of working tool 200 in addition to the afore-mentioned rotational mechanism. The mechanism consists of an SiFOW 1 which is inserted within a pathway in the mechanism (proximal portion) and tube 15 (distal portion) and is affixed and secured to the working tool by SiFOW connector 12, in connection with the working tool element 26. Rotation of one of knobs 30A or 30B advances or retracts CHTR (31) by utilizing spur gear (32) which is clasped by springs, such as spring 33A and 33B. This reciprocal linear manoeuvre of the CHTR (31), parts 26, 12 and the SiFOW are enabled while shaft 28 and high pitch screw 20 are as previously mentioned locked linearly.

As shown above, the working tool may comprise two knobs (30A and 30B), located on opposite sides of the device. The surgeon hence may tilt the tool either in clockwise or counter-clockwise directions and may operate the knob in an ergonomic and intuitive manner. Typically, circular hollow tooth rack 31 is indirectly coupled to connector 12, which locks a portion of SiFOW in place with respect to tube 15 of working tool 200 (as described hereinabove). By rotating of one or both of knobs 30A and 30B, circular hollow tooth rack 31 is displaced longitudinally which, in turn, longitudinally displaces connector 12 coupled thereto, and thereby longitudinally displaces the SiFOW coupled to connector 12. In response to rotation of one or both of knobs 30A and 30B, an offset position of the SiFOW is changed with respect to the distal end of tube 15 and of sheaths 13 and 14, such that a stable offset position of the SiFOW is maintained (until the offset controller is next adjusted). For example, distal rotation of one or both of knobs 30A and 30B offsets a position of the SiFOW by drawing the SiFOW proximally with respect to the distal end of tube 15 and of sheaths 13 and 14. In such an embodiment, the distal end of the SiFOW is retracted proximally, in order to treat a different part of tissue of the patient. Alternatively, one or both of the knobs are rotated in the opposite direction, in order to distally displace the SiFOW. The longitudinal offset displacement of the SiFOW is achieved incrementally with each rotation of one or both of knobs 30A and 30B.

Typically, the offset controller is configured to set the offset of the distal end of the SiFOW with respect to tube 15 irrespectively of a rotational position of the SiFOW. For example, when active handle 10 is partially or fully compressed and, responsively thereto, the SiFOW is rotated to a given rotation position (e.g., 30 degrees from the rotational resting position), the offset controller is able to pull the SiFOW in that rotational position, without requiring the SiFOW to first be rotated back to the rotational resting position.

It is acknowledged in this respect that the linear actuating mechanism and the rotational actuating mechanism are independently operated by the surgeon and do not affect one another, namely, the surgeon is able to rotate the SiFOW around the shaft in an accurate measure, preferably up to a maximum possible rotation angle (φₘₐₓ) without reciprocally advancing the emitting tip of the SiFOW along the shaft. Alternatively, the surgeon is able to advance and to retract the SiFOW along the shaft, preferably up to a maximum possible reciprocally-provided linear movement RLMₘₐₓ, without reciprocally rotating the emitting tip of the SiFOW around the shaft. The mechanisms are completely independent and do not affect one another other in any way, the mechanisms may be actuated separately or simultaneously. When actuated simultaneously the SiFOW's tip will manoeuvre in a path that will be combination of both.

The maximum possible rotation angle φₘₐₓ around the longitudinal axis of the shaft is possibly limited to φₘₐₓ absolute values of about 20° to about 280°, preferably φₘₐₓ absolute values of about 30° to about 80°, e.g., 72°. The maximum possible reciprocally-provided linear movement RLMₘₐₓ along the longitudinal axis of the shaft is limited to RLMₘₐₓ absolute values of about 0 mm to about 50 mm, preferably to absolute values of about 0 to about 25 mm.

It is within the scope of the invention for SiFOW 1 to emit light with an efficient and safe wavelength in the range of e.g., about 1,000 to about 1,600 nm.

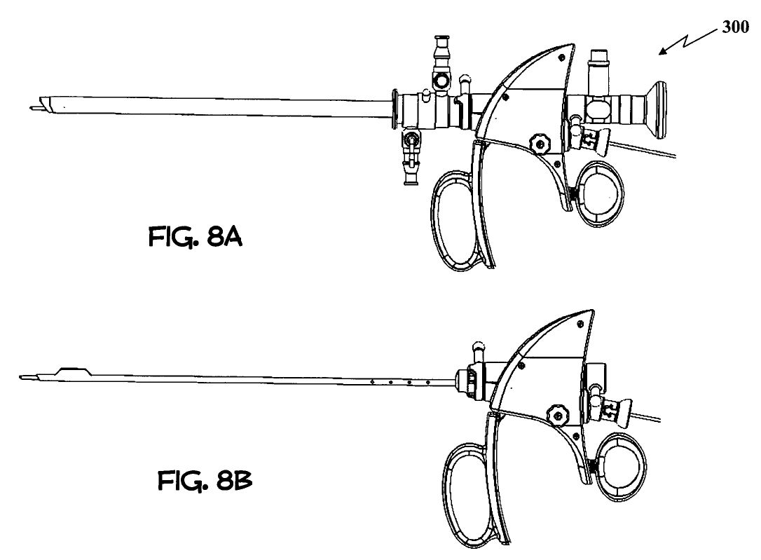

Reference is now made to FIGS. 8A and 8B, illustrating a motorized version of the aforesaid working tool (300), adapted for a motor, gear and cam shaft mechanism facilitated for rotating the SiFOW from side to side and additionally incorporating the linear mechanism introduced at working tool 200.

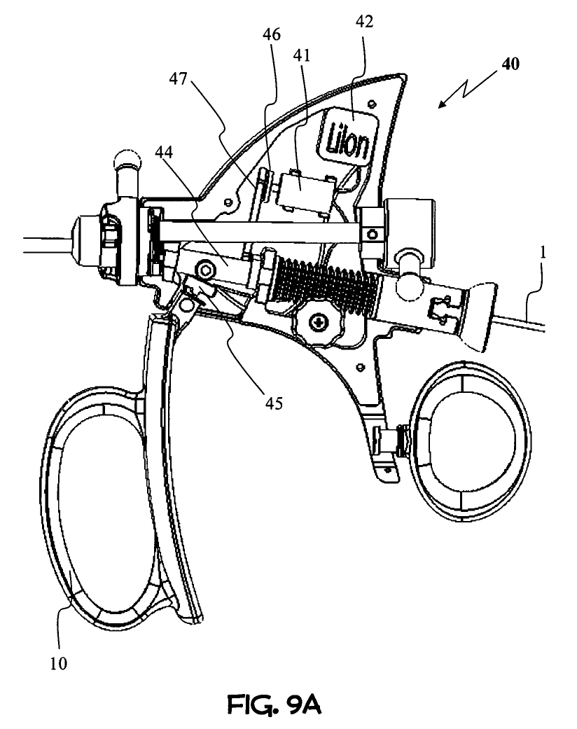

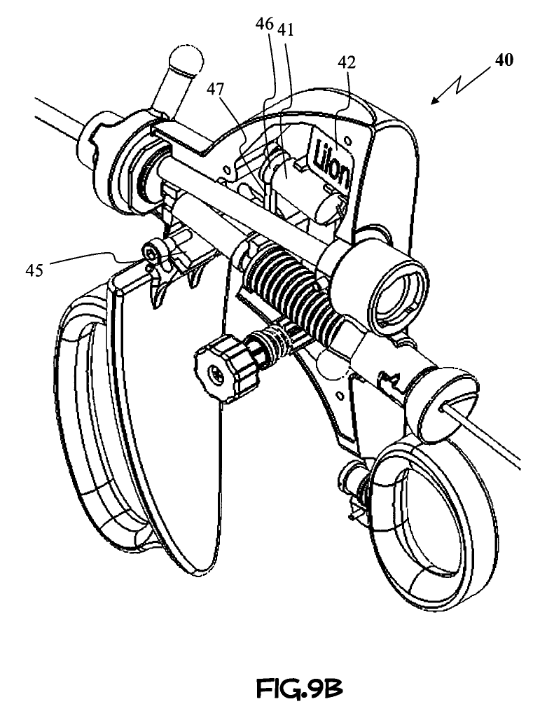

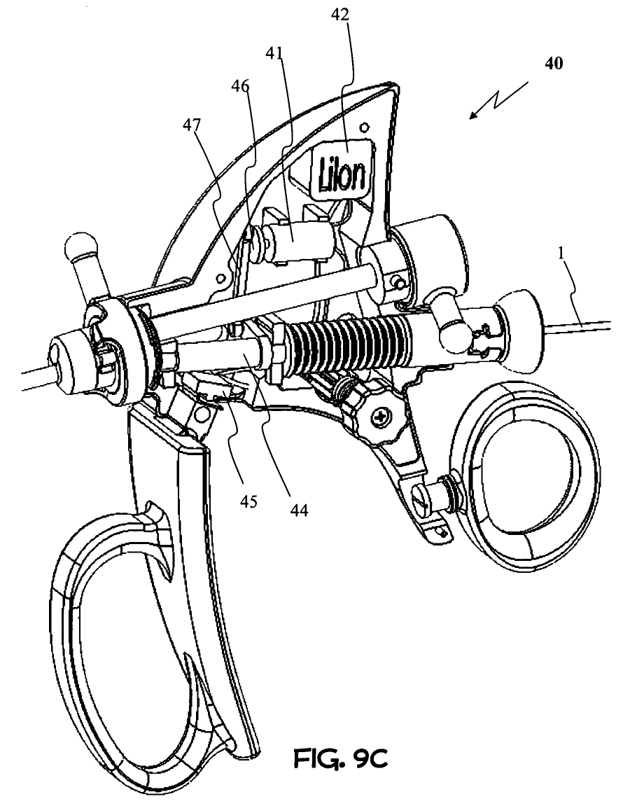

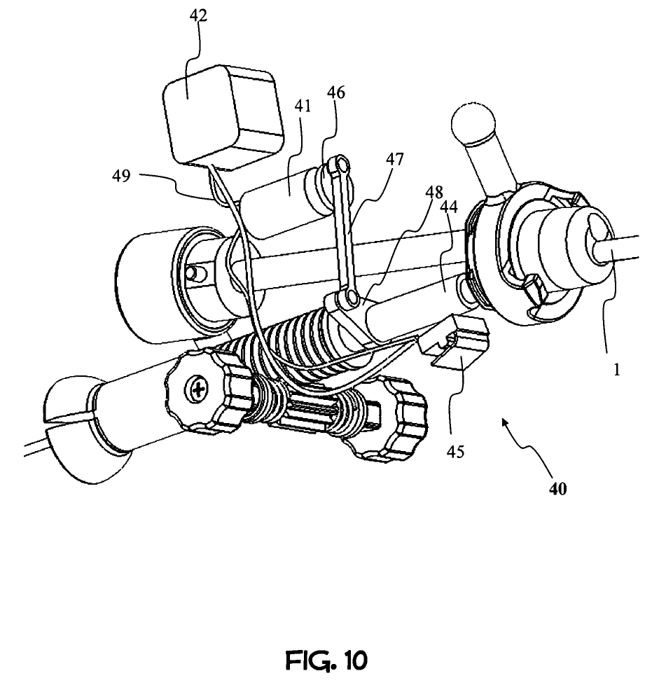

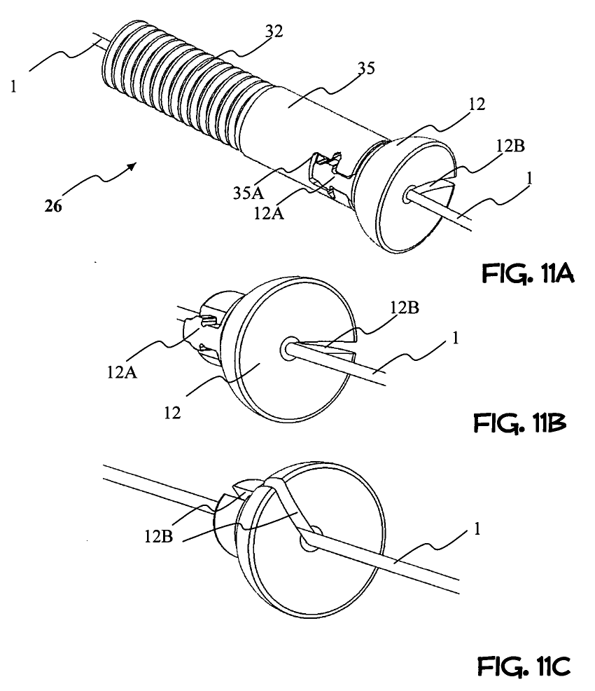

Reference is now made to FIGS. 9A, 9B and 9C. As shown in FIGS. 9A-9C, mechanism 40 comprises, in a non-limiting manner, a motor (41), optionally a motor and a gear, mechanically connected to shaft 44 by means of connecting rod assembly 47 (cam shaft), consisting of e.g., excenter 46 in connection with motor 41, rod 47, cam 48 (see FIG. 10) and shaft 44. The motor is further connected to an electrical power supply (e.g., battery 42), and to a micro-switch 45, in mechanical or electrical connection with operating handle 10. When handle 10 is held pressed, micro-switch 45 closes the electrical circuit and the motor 41 is activated, thus actuating SiFOW 1 from side to side around the shaft's axis. Here again, the SiFOW's linear positioning mechanism is completely independent and may be adjusted at any time without having any affect on the side to side movement. Micro-switch 45 may be replaced with a variator or the like, thus the rotation speed may vary in accordance with the movement or force applied to handle 10.

Reference is now made to FIG. 10, presenting the motor-facilitated, accurate and self-limited rotating mechanism (40) with its main components, namely motor 41, excenter 46, rod 47, cam 48 and shaft 44, angularly connected or otherwise affixed to SiFOW 1 through sleeve 26 (not shown) and the connector (not shown). Similarly, battery 42, electrical netting 49 and micro switch 45 are illustrated. FIG. 10 also discloses the independence of the SiFOW-motorized rotating mechanism 40 and the SiFOW-manual linear reciprocating mechanism.

Reference is now made to FIG. 11A, presenting a perspective view elongated element 26 according to one embodiment of the invention. Element 26 has an inside pathway adapted to accommodate SiFOW 1, either reversibly or irreversibly, inserted within, from its proximal end (connector 12) to its distal end (tube 15, not shown). Element 26 is characterized by a circular toothed rack (32) proximal portion and distal portion (35). The distal end of element 26 is adapted to connect SiFOW-connector 12 by means of size and shape. Hence for example, a figurative female slot 35A is designed to accommodate male-shaped connecting pin 12A of the SiFOW-connector 12. As also shown in FIGS. 11B and 11C, SiFOW-connector 12 may be designed such that the insertion of SiFOW 1 to the connector via slot 12B is easy and intuitive.

It is in the scope of the invention for the handle assemblies and tubes 15 of working tools 100, 200 or 300 to be either disposable or reusable devices, either made of at least partially flexible or rigid materials. The devices are at least partially made of plastics and FDA-approved materials, and may comprise metal ware and biocompatible alloys.

Embodiments of the present invention include a simple and cost-effective method for treating a targeted tissue located within a body cavity of a patient, especially by means of light beam facilitating vaporising/ablating and coagulating of the tissue, by a means of a laser working tool, e.g., by utilizing working tools 100, 200, 300 or a combination thereof.

The method comprises steps selected in a non-limiting manner from the following: Step (a), obtaining a working tool (e.g., 100, 200, 300) for handling and accurately actuating an SiFOW (1). The working tool has a distal portion (an elongated shaft 28 or 44) insertably located adjacent to the tissue to be treated within the body of a patient; and a proximal portion, consisting of at least one handle (e.g., handle 10), in connection with the shaft, located out of the body, held and operated by a surgeon. Step (b), providing the working tool with an actuating mechanism, namely (i) providing at least one handle with a linear-to-rotational translating means; and optionally (ii), providing at least one knob adapted for enabling the SiFOW's to move linearly. Step (c), inserting the SiFOW from the proximal portion of the working tool through its distal portion, preferably by introducing the SiFOW into a designated pathway within the actuating mechanism and/or shaft, and/or at least reversibly holding the SiFOW in position by grasping it at one or more points along the shaft, thus facilitating the SiFOW's accurate actuation. Step (d), connecting a laser light source external to the working tool to the SiFOW's proximal end. Step (e), providing the SiFOW's with means for accurate rotational actuation, by squeezing the handle and thus translating linear motion of the handle to rotational movement of the SiFOW with respect to the axis of the shaft. Optionally, step (f) is utilized, providing the SiFOW's with means for accurate linear actuation, by operating the linear actuation operating knob. Lastly, in step (g), transmitting laser light from a light outlet located adjacent to the distal end of the SiFOW and emitting light until the treatment of the area is complete.

Additionally or alternatively, step (e) is provided by facilitating a motor (41) and gear-facilitated rotating and/or a linear reciprocating (MGRL, e.g., mechanism 40) means for actuating the SiFOW. The method may comprise a further step of selecting the MGRL from a group consisting, inter alia, of direct or indirect drive, electric, pneumatic, hydraulic, piezoelectric, electromagnetic, ultrasonic, galvanic, bi-metallic, Peltier drive or any combination thereof. The method may further comprise steps of operating the motor by automatic means, semi-automatic means or manual means. The operating speed is possibly controlled by the surgeon controlled means. A further step and means of selecting the means from a group preferably consisting of handle, pedal, voice command, computer mediated means, robotic interface, or any combination thereof are also provided possible. The method may further comprise steps of focusing or defocusing the emitting tip in a predetermined manner; the defocusing is preferably provided by selecting means from a group consisting of mechanical vibration, ultrasonic, acoustic, piezoelectric motion, or other reciprocating motion. The following is a list of inventive aspects provided by some embodiments of the present invention:

1. A working tool for handling and accurately actuating a side-firing fiber optic waveguide (SiFOW); said SiFOW extends from the distal portion of the working tool through its proximal portion, having light transmitting distal end and light receiving proximal end connected to a laser light source external to said working tool; said SiFOW is utilized for treating a targeted tissue located within a body cavity of a patient, especially by means of light beam facilitated vaporization/ablation and coagulation of said tissue; said working tool having a distal portion (an elongated shaft) insertably located adjacent to the tissue to be treated within the body of a patient; and a proximal portion (handle, in connection with said shaft) located out of said body, held and operated by a surgeon; wherein said SiFOW is positioned by being at least reversibly introduced into a designated pathway and/or is at least reversibly held in position by being grasped at least at one point along the shaft, hence said shaft facilitates said SiFOW's accurate actuation; and further wherein said accurate actuation is provided by a SiFOW's actuating mechanism, comprising at least one handle, translating said handle's linear movement to SiFOW's self-limited rotation; and optionally, at least one knob providing SiFOW's self-limited adjustable linear positioning.

2. The working tool as defined in inventive aspect 1, comprising two independently-operated, self-limited (a) rotational, and (b) linear SiFOW actuating mechanisms.

3. The working tool as defined in inventive aspect 2, wherein the self-limited rotational SiFOW actuating mechanism is selected from a group of mechanisms consisting of (i) rotating screw and fixed nut; (ii) rotating nut and fixed screw; (iii) a spiral track and travelling pin, or any other means of producing rotational motion.

4. The working tool as defined in inventive aspect 3, wherein the maximum possible rotation angle φₘₐₓ around the longitudinal axis of the shaft is limited to φₘₐₓ absolute values of about 20° to about 280°, preferably φₘₐₓ absolute values of about 30° to about 60°, e.g., 45°.

5. The working tool as defined in inventive aspect 2, wherein the self-limited linear SiFOW actuating mechanism is selected from a group of mechanisms consisting of (i) spur gear mechanism; (ii) conjugated screws rotation mechanism and/or spiral tracks mechanisms, (iii) a sliding lock plate which pushes the rotational mechanism in a linear direction; (iv) a straight track and travelling pin; or any other means of producing linear motion.

6. The working tool as defined in inventive aspect 5, wherein the maximum possible reciprocally-provided linear movement RLMₘₐₓ along the longitudinal axis of the shaft is limited to RLMₘₐₓ absolute values of about 0 mm to about 50 mm, preferably to absolute values of about 0 to about 25 mm.

The working tool as defined in inventive aspect 5, wherein the said linear mechanism actuates said SiFOW by means of a spur gear and toothed rack mechanism comprising:
a) a circular tooth rack hollow gear (CTRHG) in mechanical connection with said shaft, in which the SiFOW is reversibly inserted therein;
b) a reciprocal motion knob (RMK), which is connected to and rotates said spur gear, said spur gear is mechanically coupled with said CTRHG and activates the same; and,
c) a shell housing adapted to provide mechanical support and bearing for said CTRHG, spur gear, knob and mechanisms thereof.

The working tool as defined in inventive aspect 1, wherein spring 24 enables the surgeon to temporarily and reversibly pull the SiFOW proximally out of view, thus obtaining a clear undisturbed view of the treated tissue.

The working tool as defined in inventive aspect 1, further comprising a motor and gear-facilitated rotating and/or a linear reciprocating (MGRL) means for actuating said SiFOW.

The working tool as defined in inventive aspect 9 wherein said MGRL is selected from a group consisting of: direct or indirect drive, electric, pneumatic, hydraulic, piezoelectric, electromagnetic, ultrasonic, galvanic, bi-metallic, Peltier drive or any combination thereof.

The working tool as defined in inventive aspect 9 wherein said motor is operated by automatic means, semi automatic means or manual means; and optionally wherein said operating speed is controlled by the surgeon controlled means; said means selected from a group preferably consisting of handle, pedal, voice command, computer mediated means, robotic interface, or any combination thereof.

The working tool as defined in inventive aspect 1 wherein the emitting tip of the SIFOW is either focused or defocused in a predetermined manner; said defocusing is preferably provided by means selected from a group consisting of: mechanical vibration, ultrasonic, acoustic, piezoelectric mechanism, or other reciprocating motion mechanisms.

A method for treating a targeted tissue located within a body cavity of a patient, especially by means of light beam facilitating vaporising/ablating and coagulating of said tissue, by a means of a laser working tool; said method comprising steps of:
a) obtaining a working tool for handling and accurately actuating a side-firing fiber optic waveguide (SiFOW); said working tool having a distal portion (an elongated shaft) insertably located adjacent to the tissue to be treated within the body of a patient; and a proximal portion (handle, in connection with said shaft) located out of said body, held and operated by a surgeon;
b) providing said working tool with an actuating mechanism, namely (i) providing at least one handle with a linear-to-rotational translating means; and optionally (ii), providing at least one knob adapted for enabling said SiFOW's to move linearly;
c) inserting said SiFOW from the proximal portion of the working tool through its distal portion, preferably by introducing the SiFOW into a designated pathway within the actuating mechanism and/or shaft, and/or at least reversibly holding the SiFOW in position by grasping it at one or more points along said shaft, thus facilitating said SiFOW's accurate actuation;
d) connecting a laser light source external to said working tool to the SiFOW's proximal end;
e) providing said SiFOW's with means for accurate rotational actuation, by squeezing said handle and thus translating linear motion of the handle to rotational movement of said SiFOW with respect to the axis of said shaft;
f) optionally, providing said SiFOW's with means for accurate linear actuation, by operating said linear actuation operating knob; and,
g) transmitting laser light from a light outlet located adjacent to the distal end of said SiFOW; emitting light until said treatment of said area is complete.

The method as defined in inventive aspect 13, wherein step (e) is provided by facilitating a motor and gear-facilitated rotating and/or a linear reciprocating (MGRL) means for actuating said SiFOW.

The method as defined in inventive aspect 14, further comprising a step of selecting said MGRL from a group consisting of direct or indirect drive, electric, pneumatic, hydraulic, piezoelectric, electromagnetic, ultra sonic, galvanic, bimetallic, Peltier drive or any combination thereof.

The method as defined in inventive aspect 14 comprising steps of operating said motor by automatic means, semi automatic means or manual means; and optionally wherein said operating speed is controlled by the surgeon; and selecting said means from a group preferably consisting of handle, pedal, voice command, computer mediated means, robotic interface, or any combination thereof.

The method as defined in inventive aspect 13, further comprising focusing or defocusing said emitting tip in a predetermined manner; said defocusing is preferably provided by selecting means from a group consisting of mechanical vibration, ultrasonic, acoustic, piezoelectric mechanism, or otherwise reciprocating motion mechanism.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
an intraluminal tube having a proximal end and a distal end, the tube being shaped to provide a longitudinal lumen;
an optical unit in communication with the tube and configured for viewing a view distal to the distal end of the tube;
a handle assembly coupled to the proximal end of the tube;
a treatment element configured to be disposed within the lumen of the tube, the treatment element having a proximal end and a distal end, and the treatment element having a resting position thereof in which the distal end of the treatment element extends distally from the distal end of the tube and is in the view distal to the distal end of the tube;
a spring mechanism coupled to the handle assembly; and
a user-force receiver coupled to the spring mechanism and to the treatment element, and configured to be:
displaced, with respect to the handle assembly, by a user of the apparatus, and by being displaced to: (a) apply a force to the spring mechanism, and (b) pull the distal end of the treatment element proximally, out of the view, irrespectively of a rotational position of the treatment element, and
released, and by being released, to: (a) be displaced in response to a force applied to the user-force receiver by the spring mechanism, and (b), return the treatment element to the resting position thereof.

2. The apparatus according to claim 1, wherein in the resting position, the distal end of the treatment element is configured to be disposed up to 25 mm distal to the distal end of the tube.

3. The apparatus according to claim 1, wherein the treatment element comprises a side-firing fiber optic waveguide (SiFOW) configured to treat tissue of a patient.

4. The apparatus according to claim 1, wherein the treatment element is configured to be slidably advanced through the tube from the proximal end of the tube toward the distal end of the tube.

5. The apparatus according to claim 1, wherein the treatment element is coupled to the user-force receiver, and wherein, in response to the force applied to the spring mechanism by the displacement of the user-force receiver, the treatment element is pulled proximally with respect to the tube.

6. The apparatus according to claim 1, wherein:
the spring mechanism is coupled to the user-force receiver,
the user-force receiver is coupled to a portion of the treatment element, and
in response to displacement of the user-force receiver, the treatment element is pulled proximally with respect to the tube and force is applied to the spring mechanism.

7. The apparatus according to claim 1, further comprising a motor configured to control longitudinal motion of the treatment element.

8. The apparatus according to claim 1, further comprising a motor configured to control rotational motion of the treatment element.

9. The apparatus according to claim 8, wherein the motor is configured to control longitudinal motion of the treatment element.

10. The apparatus according to claim 1, wherein the handle assembly comprises a user-grasping member configured to be longitudinally displaceable with respect to the tube.

11. The apparatus according to claim 10, wherein the treatment element is configured to be rotated about a longitudinal axis of the tube in response to longitudinal displacement of the user-grasping member.

12. The apparatus according to claim 11, wherein the treatment element is configured to be rotated no more than 360 degrees about a longitudinal axis thereof, in response to a full longitudinal displacement of the user-grasping member.

13. The apparatus according to claim 12, wherein the treatment element is configured to be rotated between 20 degrees and 280 degrees about the longitudinal axis thereof, in response to the full longitudinal displacement of the user-grasping member.

14. The apparatus according to claim 12, wherein the treatment element is configured to be rotated between 20 degrees and 80 degrees about the longitudinal axis thereof, in response to the full longitudinal displacement of the user-grasping member.

15. The apparatus according to claim 11, further comprising:
a nut coupled to the user-grasping member, and
a screw configured to be threaded through the nut, and shaped to define a longitudinal lumen for passage therethrough of the treatment element,
wherein, in response to longitudinal displacement of the user-grasping member:
the nut is configured to move longitudinally with respect to the screw and facilitate rotation thereof, and
the treatment element is configured to be rotated responsively to the rotation of the screw.

16. The apparatus according to claim 15, wherein a portion of the treatment element is coupled to the user-force receiver, wherein the user-force receiver is coupled to the spring mechanism, and wherein, in response to force applied to the user-force receiver, the user-force receiver is displaced proximally and pulls the treatment element and the spring mechanism proximally with respect to the tube.

17. The apparatus according to claim 15, wherein the screw is shaped to define a helical thread having a pitch, the pitch determining an extent of rotation of the treatment element with respect to movement of the nut.

18. Apparatus, comprising:
an intraluminal tube having a proximal end and a distal end, the tube being shaped to provide a longitudinal lumen;
a treatment element having a proximal end and a distal end, the treatment element configured to be disposed within the lumen of the tube;
a handle assembly coupled to the proximal end of the tube; and
a user offset controller coupled to the handle assembly, and configured to set an offset of the distal end of the treatment element with respect to the distal end of the tube.

19. The apparatus according to claim 18, wherein the offset controller is configured to set the offset of the distal end of the treatment element irrespectively of a rotational position of the treatment element.

20. The apparatus according to claim 18, wherein the distal end of the treatment element is configured to be disposed up to 25 mm distal to the distal end of the tube.

21. The apparatus according to claim 18, wherein the treatment element comprises a side-firing fiber optic waveguide (SiFOW) configured to treat tissue of a patient.

22. The apparatus according to claim 18, wherein a portion of the treatment element is coupled to the offset controller, and, in response to the force applied to the offset controller, a longitudinal position of the treatment element is offset with respect to the tube.

23. The apparatus according to claim 18, wherein the treatment element is configured to be slidably advanced through the tube.

24. The apparatus according to claim 18, further comprising a motor configured to control longitudinal motion of the treatment element.

25. The apparatus according to claim 18, further comprising a motor configured to control rotational motion of the treatment element.

26. The apparatus according to claim 25, wherein the motor is configured to control longitudinal motion of the treatment element.

27. The apparatus according to claim 18, wherein the user offset controller comprises:
a first gear, coupled to a portion of the treatment element;
a rotatable knob; and
a second gear, coupled to the knob and in communication with the first gear,
wherein the first gear is displaced longitudinally with respect to the second gear in response to rotation of the knob.

28. The apparatus according to claim 27, wherein a rotational axis of the first gear is substantially perpendicular to a rotational axis of the second gear.

29. The apparatus according to claim 27, wherein the offset controller is configured to offset a longitudinal position of the treatment element by longitudinally displacing, with respect to the tube, the first gear and the portion of the treatment element coupled thereto, in response to rotation of the knob.

30. The apparatus according to claim 18, wherein the handle assembly comprises a user-grasping member configured to be longitudinally displaceable with respect to the tube.

31. The apparatus according to claim 30, wherein the treatment element is configured to be rotated about a longitudinal axis of the tube in response to longitudinal displacement of the user-grasping member.

32. The apparatus according to claim 31, wherein the offset controller is configured to set the offset of the distal end of the treatment element irrespectively of a rotational position of the treatment element.

33. The apparatus according to claim 31, wherein the treatment element is configured to be rotated no more than 360 degrees about a longitudinal axis thereof, in response to a full longitudinal displacement of the user-grasping member.

34. The apparatus according to claim 33, wherein the treatment element is configured to be rotated between 20 degrees and 280 degrees about the longitudinal axis thereof in response to the full longitudinal displacement of the user-grasping member.

35. The apparatus according to claim 33, wherein the treatment element is configured to be rotated between 20 degrees and 80 degrees about the longitudinal axis thereof in response to the full longitudinal displacement of the user-grasping member.

36. The apparatus according to claim 31, further comprising:
a nut coupled to the user-grasping member, and
a screw configured to be threaded through the nut, and shaped to define a longitudinal lumen for passage therethrough of the treatment element,
wherein, in response to longitudinal displacement of the user-grasping member:
the nut is configured to move longitudinally with respect to the screw and facilitate rotation thereof, and
the treatment element is configured to be rotated responsively to the rotation of the screw.

37. The apparatus according to claim 36, wherein the screw is shaped to define a helical thread having a pitch, the pitch determining an extent of rotation of the treatment element with respect to movement of the nut.

38. The apparatus according to claim 36, wherein:
a portion of the treatment element is coupled to the offset controller, and
in response to the force applied to the offset controller, the offset controller is displaced, and responsively, the treatment element is displaced, wherein a longitudinal position of the treatment element is displaced within the screw and is offset with respect to the tube.

39. The apparatus according to claim 38, wherein the user offset controller comprises:
a first gear, coupled to a portion of the treatment element;
a rotatable knob; and
a second gear, coupled to the knob and in communication with the first gear,
wherein the first gear is displaced longitudinally with respect to the second gear in response to rotation of the knob.

40. The apparatus according to claim 39, wherein a rotational axis of the first gear is substantially perpendicular to a rotational axis of the second gear.

41. The apparatus according to claim 39, wherein the portion of the treatment element is disposed within the first gear, and wherein the offset controller is configured to offset the longitudinal position of the treatment element by longitudinally displacing with respect to the tube the first gear and the treatment element disposed within the first gear, in response to the rotation of the knob.

42. A method, comprising:
advancing through a lumen a tube having a proximal end and a distal end, the tube being configured to be coupled to a treatment element having a proximal end and a distal end, and the treatment element having a resting position thereof in which the distal end of the treatment element extends distally from the distal end of the tube;
viewing a view distal to the distal end of the tube;
irrespectively of a rotational position of the treatment element, retracting the distal end of the treatment element to a site proximal to the view distal to the distal end of the tube, while applying a force to a spring mechanism in communication with the treatment element by pulling a force receiver coupled to the spring mechanism; and
returning the treatment element to the resting position thereof by releasing the force receiver.

43. The method according to claim 42, further comprising advancing through the tube the treatment element to the resting position thereof, and placing the distal end of the treatment element distally to the distal end of the tube and in the view distal to the distal end of the tube following the advancing.

44. The method according to claim 43, wherein advancing the treatment element through the tube comprises advancing the treatment element through the tube until a distal end of the treatment element is disposed up to 25 mm distal to the distal end of the tube.

45. A method, comprising:
advancing through a lumen a tube having a proximal end and a distal end, the tube being configured to be coupled to a treatment element having a proximal end and a distal end; and
offsetting a position of the treatment element from a first position to a second position with respect to the distal end of the tube by manipulating an offset controller that secures the treatment element at the second position.

46. The method according to claim 45, wherein offsetting the position of the treatment element comprises offsetting the position of the treatment element irrespectively of a rotational position of the treatment element.

47. The method according to claim 45, further comprising advancing through the tube the treatment element to the first position, and placing the distal end of the treatment element distally to the distal end of the tube and in the view distal to the distal end of the tube following the advancing.

48. The method according to claim 47, wherein advancing the treatment element through the tube comprises advancing the treatment element through the tube until a distal end of the treatment element is disposed up to 25 mm distal to the distal end of the tube.
